(19) United States
(12) Patent Application Publication
(10) Pub. No.: US 2009/0011442 A1
(43) Pub. Date: Jan. 8, 2009

(54) TB diagnostics based on mycobacterium tuberculosis excretory secretory antigens and their specific immunoglobulins

(76) Inventor: Harinath C. Bhaskar, Sewagram (IN)

Correspondence Address: DR. HARINATH C. BHASKAR, 43 Independence Drive, Hillsborough, NJ, 08844, US

(21) Appl. No.: 12/009,098

(22) Filed: Jan. 16, 2008

Related U.S. Application Data
(60) Provisional application No. 60/885,398, filed on Jan. 17, 2007.

(51) Int. Cl. G01N 33/53; C07H 21/00; C07K 16/00

(52) U.S. Cl. 435/7.32; 536/23.7; 530/388.4; 530/389.5; 435/7.92; 435/7.94

(57) ABSTRACT

The present invention relates to isolation and identification of novel M. tuberculosis excretory secretory antigen proteins released in vitro/in vivo, raising the specific immunoglobulins and their use as diagnostic reagents for tuberculosis. The invention further relates to a combination of antigens and immunoglobulins for effective detection and immunomonitoring of pulmonary or extrapulmonary Mycobacterium tuberculosis in children and adults by different immunological methods. The invention also relates to detection of tuberculosis with HIV coinfection. Further the invention also shows potential of mycobacterial metallo serine protease (SEVA TB ES-31 antigen) as drug target for screening potential antitubercular drugs.

This application claims priority from Provisional U.S. Patent Application Serial No. 60885398, filed on Jan. 17, 2007.

### BACKGROUND

Tuberculosis (TB) is a chronic, infectious disease caused by infection with Mycobacterium tubercle bacillus. Infection may often be asymptomatic, but could lead to disease, producing pulmonary or extrapulmonary lesions which might result in severe debilitation or death. TB is believed to be the second leading killer with one death per minute (RNTCP, India) among the infectious diseases. It can spread through air when infected individuals cough, sneeze, or even merely speak to one another. The current rate of TB infection has been estimated at one person per second. As estimated by CDC, 10 to 15 million Americans are currently affected by TB with potential to develop into active TB at some point in future.

Mycobacterium tuberculosis has been declared as global emergency by WHO in 1993 and has been identified as a category C agent as per Bioterrorism guidelines issued by Center for Disease control (CDC) and National Institute of Allergy and Infectious diseases (NIAID) (ref. MMWR 2000; 49) (No. R R-4).

Tuberculosis diagnosis has been identified as a key area of research and development by National Institute of Health. Tuberculosis remains a significant public health problem in developing countries and on upward trend is seen due to advent of HIV infection and multidrug resistant strains of M. tuberculosis. HIV and TB form a lethal combination each speeding up the others' progress. TB with HIV co-infection has been a lethal combination causing one death out of 3 patients with AIDS.

About 1.7 billion or one third of World's population have been infected with Mycobacterium tuberculosis (TB India RNTCP Status report, 2001). Globally 8.8 million new cases with 1.7 million deaths have been reported due to tuberculosis in 2003. One third of 40 million HIV infected patients are coinfected with TB. The WHO declared tuberculosis as a Global Emergency in 1993.

Human immunodeficiency virus (HIV) infection is a potent risk factor for tuberculosis (TB), with one third rise in TB associated with HIV. Statistics indicate that TB is the leading killer with one death out of 3 patients with AIDS. The incidence of TB is found to be 30% in HIV infected patients. Studies have shown that the TB-HIV co-infection rate in India is as high as 60%. The present invention relates to early detection of TB in HIV patients and thus helps in better clinical management. Regular TB screening for AIDS patients is highly recommended.

Extrapulmonary tuberculosis constitutes about 15-20% of all cases of TB while it accounts for more than 50% of cases in HIV positive individuals. The present invention relates to detection of extrapulmonary tuberculosis based on blood sample, thus obviating the need for invasive procedures to collect specimens.

Early detection of MDR-TB (Multi Drug Resistant TB) and XDR-TB (Extensively Drug Resistant TB) and assessment of compliance in drug treatment are important for successful TB control program.

### Diagnostics:

Presently diagnosis of tuberculosis largely depends upon clinical, radiological, cytological and bacteriological examinations. Direct microscopy of sputum for bacilli is not sensitive, and not helpful in extrapulmonary TB and childhood tuberculosis, where sputum is not available. Culture method is cumbersome and takes lengthy periods of time; for example, a currently marketed diagnostic product touts itself as a landmark system because it shortens TB recovery in culture from 42 days to approximately 10 to 14 days. Nucleic acid amplification tests are similarly problematic, in that they require specialized laboratory facilities, skilled personnel and costly (Pai M, et al. Lancet Infect Dis. 2003, 3:633-43). Serological diagnosis based either on the detection of antibody or antigen, however, is a comparatively simple diagnostic tool with flexibility to adopt to small laboratory in field condition. The immunogenic products of the living organisms obtained through their in vitro maintenance or from in vivo fluids, the so called ‘excretory secretory’ antigens represent promising candidates for developing immunodiagnostics. Secreted proteins are likely to provide the first stimulus in vivo for the humoral and cellular responses to mycobacteria and may be valuable in serological diagnostic test. Isolation and partial characterization of some of the culture filtrate proteins of mycobacterium tuberculosis have been reported (Nagai, et al. Infect. Immun. 1991, 59:372-382; Anderson, P et al. Infect. Immun. 1991, 59:1905-1910). Earlier studies were more of basic in nature and of academic interest, concentrating on biochemical characterization of proteins secreted on different days during growth of bacilli.

### FIELD OF INVENTION AND RELATED ART

001 Mycobacterium tuberculosis is a global health problem requiring simple diagnostic test for successful TB treatment and control programs.
002 Many components of mycobacteria comprising proteins, free or complexed with carbohydrates or lipids of cell membrane, cytoplasmic proteins and enzymes from living or dead cells interact with the immune system of infected host organism eliciting humoral immune responses in the form of specific immunoglobulins.
003 Excretory secretory (ES) proteins by live mycobacterial cells are likely to provide the first stimulus in vivo for the humoral or cellular responses. Hence the ES proteins obtained from short term cultures in synthetic medium should be ideal targets for study and developing diagnostic tests for detecting the presence of live and ongoing TB infection, as detection of live infection is important in arresting transmission of the disease in the community.
004. The invention relates to field of diagnostics providing number of novel excretory secretory protein antigens and specific immunoglobulins useful in the diagnosis and monitoring of pulmonary and extrapulmonary tuberculosis in adults and children.
005. Several immunological methods for detecting mycobacterial antigens such as enzyme linked immunosorbent assay (ELISA) and radioimmunoassay (RIA) have been mentioned as possible alternatives to microscopy (Daniel, Review of Infectious Diseases Vol. II Supplement 2, March-April 1989 p 5471-5478).
006 Antibody detection assay (immune response of infected host) by ELISA has been more common for number of infectious diseases using specific antigens. However, these assays are influenced by disease prevalence rate and cross reactivity with other diseases. Immunodetection of circulating free or immune complexed antigen during active infection is independent of host's immune response and thus antigen assay could be a better diagnostic marker.
007 Secreted antigens are highly immunodominant and measurement of humoral response directed against them gives an indication of active infection.
008 A large variability in the seroreactivity of secreted antigens has been reported depending on antigen employed, the immunoglobulin class measured, stage of disease, strain variation and the deviation in the antibody response among the different ethnic groups. (Bothamley GH. Eur. Respr. J 1995; 8:6765-6885).
   There is a need for identifying, characterizing the ES antigens and study their seroreactivity.

The Present Invention Relates to Isolation, Identification and Characterization of
009 A 31-32 kDa mycobacterial Zn containing serine protease (Seva ES-31) from Mycobacterium tuberculosis H₃₇Ra strain bacilli culture filtrate by ammonium sulphate precipitation, SDS—PAGE and Fast Protein Liquid Chromatography using cation exchange resin column.
010 Seva ES-41 antigen protein (41-42 kDa) by TCA precipitation of culture filtrate antigen protein isolated from M. tb H₃₇Ra bacilli culture medium followed by FPLC using Gel filtration and cation exchange chromatography.
011 Seva ES-43 antigen protein (43-44 kDa) was isolated from M. tb H₃₇Ra bacilli culture medium by ammonium sulphate precipitation, SDS-PAGE followed by FPLC using anion exchange chromatography.
012 Seva EST-6 antigen protein fraction was isolated from M. tb H₃₇Ra bacilli culture medium_by TCA precipitation of culture filtrate antigen followed by SDS-PAGE fractionation which was found to contain 38-39 kDa and 41-42 kDa protein antigens.

### The Present Invention Relates to Seroreactive Studies Showing

013 That Seva ES-31 was more reactive in pulmonary tuberculosis, tuberculous lymphadenopathy and tubercular meningitis, while Seva ES-41 antigen was more reactive in abdominal TB and bone and joint TB.
014 Elevated levels of Seva ES-31 and Seva ES 41 antigens in sera of these patients with different forms of tuberculosis such as lung TB, abdominal TB and Bone and joint TB.
015 That of fresh, relapse and chronic cases of TB, Seva ES-43 was primarily recognized by serum antibodies in relapse cases.
016 Seva ES-31, ES-41 and ES-43 showed heterogeneous antibody response when tested individually at different stages of disease progression in tuberculosis. Possibly a combination of these antigens should help as a broad spectrum diagnostic reagent for all stages and forms of tuberculosis.
017 Some of the sputum positive tuberculosis cases did not show the presence of antibody to SEVA ES-31 antigen, however showed presence of antigens when affinity purified antibody (anti ES-31) was used in Sandwich ELISA. Thus antigen assay may be useful as adjunct test.
018 A follow up study of immune status during ATT course of tuberculosis patients, revealed a gradual decrease in the antigen followed by antibody to Seva ES-31 antigen.
019 Specific immunoglobulins to Seva ES-31, ES-43 and EST-6 were isolated from ES/DSS (Detergent Soluble M. tb H₃₇Ra cell Sonicate) antigen immunized goat anti sera.
020 Specific immunoglobulins were coupled to sepharose 4B to isolate different antigens from culture fluid/TB bacilli by affinity chromatography.
021 HIV patients normally exhibit immunosuppression and thus TB and HIV coinfected will not show good immunoglobulin response to specific TB antigens.
022 Assay of free and immune complex antigen has been found to be useful in detection of TB with HIV coinfection.
023 In general and in the broadest aspect, the antibody or antigen based assay involve providing a sample of serum from a patient infected with or suspected of being infected with M. tuberculosis and analyzing the same for the presence of the tuberculous antibodies or circulating free or immune complexed antigens by means of ELISA or other immunological techniques using one or more substantively purified ES protein antigens (Cocktail of antigens) and specific immunoglobulins, raised and isolated against these cocktail of antigens, thus rendering diagnosis of pulmonary and extrapulmonary tuberculosis, relates to this invention.
024. The antibody—based assay of the present invention employs an indirect ELISA, where mycobacterial cocktail of antigens Seva ES-31, EST-6 and ES-43 coated on the solid phase is contacted with a serum sample. The contacting step is conducted under conditions which allow antigen—antibody binding to occur if the serum contains antibody to any one or all of the cocktail antigens Seva ES-31, EST-6 and ES-43. The antigen—antibody complex on the solid phase is then detected as an indication of the presence of anticocktail ES antigen antibodies in the serum sample. Detection is a multi step process.
   The solid phase is contacted with a secondary antibody i.e. anti human IgG conjugated to an enzyme penicillinase/peroxidase which is capable binding to the serum anti cocktail antibody if present on the solid phase. Then the antigen—antibody—secondary antibody—enzyme is contacted with suitable substrate and the enzymatic reaction is visualized or measured.
   Tables 1 and 2 show detection of tuberculous antibody to a cocktail II of Seva ES-31, ES-41 and ES-43 antigens in tuberculosis and Table 3 shows the reactivity pattern of different combinations of antigens by immunoblotting in pulmonary tuberculosis and Table 4 shows comparative analysis of seroreactivity of cocktail antigen by ELISA and immunoblotting in PTB.
025 The antigen based assay of the present invention is a double antibody assay (Sandwich ELISA or Capture ELISA) carried out by combining a serum sample of patient infected with or suspected of being infected with M. tuberculosis, containing antigen with affinity purified anti cocktail Seva ES-31, ES-43 and EST-6 antigens antibody. Anti DSS-Ag antibody labeled with penicillinase/peroxidase provides a detectable signal with the presence of circulating one or all free cocktail antigens which have been complexed with specific immunoglobulin.
026 For detecting immune complexed antigen, serum samples were pretreated with Glycine HCL buffer followed by heating at 65° C. to release antigen and then sandwich ELISA was performed as above.

Table 5 shows comparative reactivity of M. tb. H₃₇Ra anti ES-31 antibody and Seva ES antigen cocktail III antibody (anti ES-31, anti ES-43 and anti EST-6) for detecting circulating free and immune complexed antigen in pulmonary tuberculosis by sandwich ELISA and Table 6 shows the comparative reactivity of M. tb. H₃₇Ra anti ES-31 antibody and Seva cocktail ES antigen antibody for detecting circulating free and immune complexed antigen in different stages of pulmonary tuberculosis. Table 7 shows the detection of antibody, free and immune complexed ES-31 antigen in TB with HIV co-infection. Table 8 shows immunomonitoring by detection of antibody and ES-31 antigen during antituberculosis treatment and Table 9 shows comparative evaluation of assay of ES-31 antigen (published) verses Seva cocktail III A ES antigen (unpublished) in TB with HIV co-infection and Tables 10 & 11 show usefulness of SEVA cocktail III of ES antigens (ES-31+ES-43+EST-6) and their specific antibodies (cocktail III B) in detection of antibody and antigen (Free and IC-antigen) respectively in sputum/culture positive and clinically suspected cases of extrapulmonary tuberculosis.

Tables 12 & 13 show usefulness of SEVA Cocktail III A of ES antigens (ES-31+ES-43+EST-6) and their specific antibodies (cocktail III B) in detection of antibody and antigen (free & IC-antigen) respectively in bacteriologically sputum+ve confirmed cases and clinically suspected cases of pulmonary tuberculosis.

Table 14 shows usefulness of SEVA TB ES-6 antigen in detection of TB in contact cases (latent infection).

Table 15 shows summary of antibody and antigen detection (Ag+IC-Ag) using different SEVA TB ES antigen cocktails or their specific antibody cocktails in pulmonary, extra pulmonary and HIV-TB coinfection.

Table 16 shows biochemical characterization of ES-31 and ES-43 as glycoproteins and ES41 as lipoprotein.

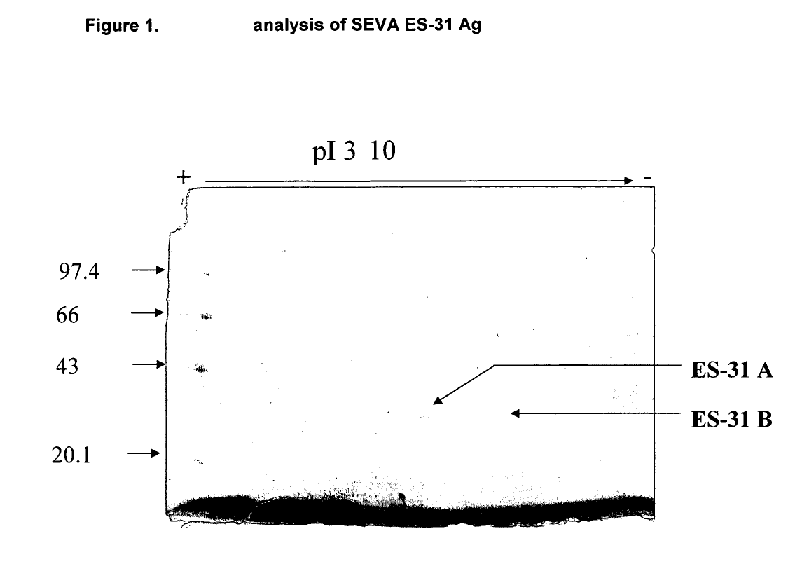

Table 17 shows purification of ES-31 antigen by various steps such as Salt precipitation, SDS-PAGE and FPLC gave 3096 fold purification and further separation by 2D gel gave two prominent bands A & B (FIG. 1). Predominent band A showed the presence of Zn containing serine protease activity.

### Inhibition of Serine Protease by Protease Inhibitors and Anti Tubercular Drugs.

Enzyme activity was inhibited by pefabloc, 3,4-Dichloroisocoumarin, phenyl methyl sulphonyl fluoride, EDTA and 1,10 phenanthroline showing ES-31 antigen is a zinc containing serine protease (Table 18). Antitubercular drugs namely Isoniazid, Ethambutol and Streptomycin inhibited serine protease activity in vitro and inhibition of bacillary growth was also observed on LJ slant and in Sautons' broth (Table 19 & 20). Amongst the drugs tested, isoniazid inhibited 85% serine protease activity thus SEVA TB ES-31 may have potential as drug target for screening anti tubercular drugs.

### Molecular Analysis of Serine Protease

LCMS-T analysis: LCMS-T analysis of predominant gel spot, ES-31 A (pl 6.3) protein was done at The Centre for Genomic Application, New Delhi. Mascot Database search result in Bacteria (Eubacteria) taxonomy has shown two conserved amino acid sequences (1) TCAFIDAEHALDPIYAKK, (2) KAEIEGEIGDSHMGLAARM.

On blasting these peptide sequences against M. tuberculosis H₃₇Ra genome.sequence showing match with Rec A (Recombinase A) protein (Mol.Wt.=85.258 kDa) with 66% identity.

Amino Acid Sequence of Rec A Protein of M. tuberculosis H₃₇Ra Strain:

### Sequence Listing Enclosed on CD on a Computer Readable Format

### Nucleotide Sequence for the Amino Acid Sequence of Rec A Protein by TBLASTN 2.2.17

### Sequence Listing Enclosed on CD on a Computer Readable Format

The invention will now be described with reference to the following examples:

### EXAMPLE 1

### Sera

Human sera belonging to different groups viz., pulmonary TB, extrapulmonary TB, TB with HIV co-infection, non-tuberculous disease control and healthy control were used for the analysis of tuberculous antibodies and antigen in the present invention. Blood samples were collected from the indoor and outdoor patients attending the Departments of Medicine and Orthopaedics of Kasturba Hospital, Mahatma Gandhi Institute of Medical Sciences, Sevagram India and also from patients attending District Tuberculosis Centre, Civil Hospital, Wardha India. Intravenous blood samples were collected in sterile vials and allowed to clot at room temperature for 30 min.

The serum was separated by centrifugation at 4° C. and stored at −20° C. with sodium azide (0.01%) as preservative.

### EXAMPLE 2

### Preparation of Mycobacterium Excretory-Secretory Antigen (M. tb ES Ag).

The Mycobacterium tuberculosis H₃₇Ra strain was provided by-Tuberculosis Research Centre, Chennai, India for research purposes. The bacilli were subcultured from Lowenstein Jenson (L-J) slants in liquid synthetic Sauton's medium for isolation of mycobacterial excretory secretory proteins. The bacilli were grown on the thyroxine supplemented L-J medium for a period of 2 weeks at 37° C. in a bacteriological incubator (Prototech). The loopful of bacilli (1×10⁴ bacilli/ml) was scrapped from the L-J slant and inoculated in 10 ml thyroxine supplemented Sauton's medium in culture bottles incubated at 37° C. for 3 days with shaking followed for another 7 days incubation in 100 ml Sauton's medium taken in 500 ml conical flask.

For isolation of M. tb ES antigenic protein the bacilli were separated from the medium by filtration through Whatmann 3 filter paper followed by sterile filtration through Seitz filter followed by 0.45 μm Maxflow membrane filter. Further the ES antigenic proteins were concentrated using spectrophor dialysing membrane with a molecular weight cut off 10-14 kDa and stored at −20° C.

### EXAMPLE 3

### Purification of M. tb ES Protein Antigen by Ammonium Sulphate Precipitation Followed by SDS-PAGE and FPLC

The ammonium sulphate soluble fraction of M. tb ES antigenic protein (ESAS) was isolated by adding ammonium sulphate in installments to a final concentration of 50% at ice cold temperature. The clear supernatent was then dialysed extensively against 0.01M PBS, pH 7.2 and concentrated by ultrafiltration.

M. tb ESAS Ag was diluted with an equal volume (1:1) of SDS sample buffer in a boiling water bath for 3 minutes, applied in wells and analysed by SDS-PAGE. The samples were stacked at a constant current of 20 mA and separated at 25 mA until the tracking dye (bromophenol blue) reaches 12 cm length in the resolving gel.

After the electrophoresis the proteins from the gel were eluted, by electroelution. The gel was sliced horizontally into 12 slices at 1 cm intervals. The 7^{th} slice showed two bands stained by silver stain. The proteins from the 7^{th} gel slice having antigenic activity were recovered by electroelution into Tris-glycine buffer (pH 8.3). The eluant was dialysed extensively for complete removal of Tris base against 0.01 M PBS, pH 7.2 and designated as M. tb ESAS-7 antigen.

ESAS-7 was further fractionated by fast-protein liquid chromatography (FPLC) on a 1 ml Resource ‘S’ cation exchange column at a flow rate of 1 ml/min with a 0 to 100% NaCI gradient. The sixth peak ESAS-7F protein (Seva ES-31) fraction confirmed by SDS-PAGE followed by silver staining having 31-32 kDa protein was collected, pooled and concentrated by freeze drying. The protein content was estimated by Lowry's method. Biochemical characterization studies showed glycoprotein nature of Seva ES-31 with serine protease activity.

### EXAMPLE 4

### Production and Isolation of Antibodies to ES Proteins in Goat

Polyclonal antibodies were raised in goat by immunising with 500 μg of ESAS antigen/DSS—Ag. The immune serum was collected 10 days after the last dose and on subsequent two days. The Ig fraction was isolated by 35% ammonium sulphate precipitation under ice. The IgG fraction was isolated by DEAE-cellulose column chromatography. The unabsorbed fraction (IgG) was collected, pooled and lyopholized.

Monospecific anti ES-31 antibodies were isolated from hyper immune anti ES goat sera by affinity chromatography using M. tb ES-31 Ag coupled CNBr-activated sepharose—4B. Similarly monospecific antibodies were isolated for Seva ES-43 and Seva EST-6.

### EXAMPLE 5

### Isolation of ES Antigen Equivalents from Tubercle Bacilli

Immunodiagnostically useful M. tuberculosis H₃₇Ra protein antigens ES-31, ES-43 and EST-6 were isolated from mycobacterial detergent soluble (DSS) antigen using monospecific antibodies prepared in Example 4 coupled to CNBr Sepharose-4B beads.

### EXAMPLE 6

### Conjugation of Proteins to Enzyme

Conjugation of antihuman IgG or affinity purified anti Seva ES antigen IgG with enzyme penicillinase was achieved by one step Avramea's method using glutaraldehyde. The volume was made to 1 ml with 0.25 M Sodium Phosphate Buffer, pH 7.2. The conjugate was then dialyzed against 0.01 M SPB for 24 hr with 3 changes of buffer, centrifuged at 400 g and the supernatant was stored at 4° C. with 0.01% sodium azide as preservative.

### EXAMPLE 7

### Enzyme-Linked Immunosorbant Assay (ELISA)

Substrate for penicillinase ELISA was prepared by dissolving 150 mg of soluble starch in 27.5 ml of 0.25 M SPB, pH 7.2 by indirect heating. After cooling the starch solution to room temperature, 10.64 mg of penicillin ‘V’ and 100 μl of 0.08 M iodine in 32 M potassium iodide solution was added. The substrate was prepared fresh before use.

### a) Indirect ELISA (Penicillinase Enzyme Immunoassay)

Indirect ELISA was carried out for detection of tuberculous antibody in sera. The cellulose acetate membrane (CAM) squares (5×5 mm) fixed on plastic strips (5×70 mm) were used as the solid support for antigen coating.

The CAM sticks were coated with 5 μl of optimally diluted M. tb cocktail ES protein antigen in 0.05 M SPB (Sodium Phosphate Buffer), pH 7.2 and air dried. The unbound sites on CAM were saturated by incubating the CAM sticks in 3% gelatin in same buffer at 37° C. for 2 hours. After washing with PBS/T 5 times, the sticks were incubated with 0.5 ml of optimally diluted (1:600 and serial two fold) sera in PBS/T (Phosphate Buffer Saline/tween 20) at 37° C. for 1 hour. The sticks were again washed for 5 times with PBS/T and incubated with 0.5 ml of optimally diluted (1:1000) antihuman IgG penicillinase conjugate in PBS/T (Phosphate Buffer Saline/tween 20) at 37° C. for 30 min. After final washing with PBS/T PBS/T (Phosphate Buffer Saline/tween 20) for 9 times, the immune reaction was observed by incubating the sticks in 0.5 ml of starch-iodine-penicillin ‘V’ substrate at 37° C. for 25 min. The decolorization of the blue colored substrate denoted a positive reaction while negative reaction was confirmed by persistance of blue color.

### b) Indirect Plate Peroxidase ELISA

The ES antigen was diluted to optimum concentration with carbonate buffer (pH 9.6) before being used to coat the solid phase. A 96-well flat bottomed microtiter plate (Tarsons, India) was used as the solid phase. Fifty microliters of the diluted antigen was added to each test well and the plates were incubated overnight at 4° C. The plates are subsequently aspirated and incubated for 2 hours at 37° C. with 100 μl/well of 2% Bovine Serum Albumin (BSA) in carbonate buffer (pH 9.6) to block the non specific binding sites. The plates were washed three times with PBS/T and the test sera from patients with confirmed/suspected tuberculosis as well as from healthy controls were each diluted (1:100 and serial two fold) in PBS/T (Phosphate Buffer Saline/tween 20) and fifty microliters of the diluted solution was added to each well in duplicate and incubated at 37° C. for one hour. The plates were washed 3 times with PBS/T (Phosphate Buffer Saline/tween 20) and horse radish peroxidase conjugated rabbit anti-human IgG (commercial reagent) was added to each well at a dilution of 1:2000. The plates are incubated at 37° C. for one hour and washed again. Fifty microliters of a freshly prepared orthophenylenediamine (OPD) and hydrogen peroxide in citrate buffer (pH 5) was used for the enzymatic reaction. The plates were incubated for 15 minutes in dark at room temp. The color reaction was stopped by adding 50 μl of 5N hydrochloric acid. The anti mycobacterial antibody of each well was measured at wavelength 492 nm using ECIL Micro Scn MS-560 SA ELISA Reader.

### c) Sandwich ELISA (Penicillinase/Peroxidase)

Sandwich ELISA was carried out for detecting circulating mycobacterial antigen levels in the serum samples.

The CAM sticks were coated with 5 μl of optimally diluted affinity purified specific anti cocktail ES Ag IgG in 0.05 M SPB, pH 7.2 and air dried. The unbound sites on CAM sticks were saturated with 3% gelatin in the same buffer. After incubation for 2 hr at 37° C., the sticks were washed with PBS/T (Phosphate Buffer Saline/tween 20) for 3 times and incubated further with 0.5 ml of optimally diluted serum samples (1:300 and serial two fold) in PBS/T (Phosphate Buffer Saline/tween 20) at 37° C. for 1 hr. After washing the sticks 3 times with PBS/T (Phosphate Buffer Saline/tween 20), 0.5 ml of optimally diluted affinity purified anti ES antigen/anti DSS—Ag IgG antibody penicillinase conjugate (1:1000) was added and incubated for 1 hr at 37° C. After final washing (5 times with PBS/T) the immune reaction was observed by incubating the sticks with 0.5 ml of freshly prepared starch-iodine-penicillin ‘V’ substrate. The disappearance of the blue colour at least 5 minutes before the negative control, denoted a positive reaction.

The assay may be done using enzyme peroxidase conjugated to affinity purified antibodies.

### EXAMPLE 8

### Seva ES-31 As Drug Target

Enzyme activity was inhibited by pefabloc, 3,4-Dichloroisocoumarin, phenyl methyl sulphonyl fluoride, EDTA and 1,10 phenanthroline showing ES-31 antigen is a zinc containing serine protease. Antitubercular drugs namely Isoniazid, Ethambutol and Streptomycin inhibited serine protease activity in vitro and inhibition of bacillary growth was also observed on LJ slant and Sautons' broth. Amongst the drugs tested, isoniazid inhibited 85% serine protease activity thus SEVA TB ES-31 may have potential as drug target for screening anti tubercular drugs. The invention also shows the potential of Seva TB ES-31 antigen, a mycobacterial metallo serine protease as drug target thus its usefulness for screening antituberculosis drugs.

### Tables:

TABLE 1
Seroreactivity of ES-31 and cocktail antigen (ES-31 + ES-41) by
stick Indirect Penicillinase ELISA.

| | No. of | No. showing Positive reaction | |
|---|---|---|---|
| | Sera | for antibody detection by | |
| Group (Sera) | tested | ES-31 | ES-31 + ES-41 |
| Pulmonary TB | 25 | 23 (92%) | 23 (92%) |
| Tuberculous Lymphadenopathy | 25 | 22 (88%) | 22 (88%) |
| Tuberculous Meningitis | 10 | 9 (90%) | 9 (90%) |
| Abdominal TB | 27 | 19 (70%) | 22 (81.5%) |
| Bone and Joint TB | 26 | 18 (69.2%) | 22 (84.6%) |
| Disease control | 46 | 4 (8.7%) | 4 (8.7%) |
| Healthy control | 30 | 2 (6.6%) | 2 (6.6%) |

*Sera showing positive reaction at 1:600 serum dilution.

TABLE 2
Detection of tuberculosis antibody to a cocktail of antigens ES-
31, ES-41 & ES-43 by ELISA in anti ES-31 IgG positive and negative
PTB cases.

| | No. | No. positive* to cocktail |
|---|---|---|
| Group | screened | antigen |
| Pulmonary Tuberculosis | | |
| Anti ES-31 IgG positive | 20 | 20 (100%) |
| Anti ES-31 IgG negative | 7 | 6 (86%) |
| Healthy control | 10 | — |
| Disease control | 5 | — |

*Sera showing positive reaction at 1:600 serum dilution.

TABLE 3
Reactivity pattern to different combination of antigens by
immunoblotting in pulmonary tuberculosis.

| | S+, | S+, | | S−, | S−, | |
|---|---|---|---|---|---|---|
| | anti ES-31 | anti ES-31 | | anti ES-31 | anti ES-31 | |
| | IgG +ve | IgG −ve | | IgG +ve | IgG −ve | |
| Reactivity to | (n = 15) | (n = 5) | Total (n = 20) | (n = 5) | (n = 2) | Total (n = 7) |
| 31 | 12 | — | 12 | 2 | — | 2 |
| 41 | 6 | 1 | 7 | 1 | — | 1 |
| 43 | 11 | 3 | 14 | 2 | 1 | 3 |
| 31/41 | 12 | 1 | 13 | 2 | — | 2 |
| 31/43 | 13 | 3 | 16 | 3 | 1 | 4 |
| 41/43 | 11 | 3 | 14 | 2 | 1 | 3 |
| 31/41/43 | 13 | 3 | 16 | 3 | 1 | 4 |

S +ve - Smear-positive PTB
S−ve - Smear negative PTB

TABLE 4
Comparative analysis of seroreactivity of cocktail antigen by
ELISA and Immunoblotting in pulmonary tuberculosis.

Positivity to
cocktail antigen in

| Group | No. screened | Blotting | ELISA |
|---|---|---|---|
| Pulmonary TB | | | |
| Sputum positive | 20 | 16 (80%) | 19 (95%) |
| Sputum negative | 7 | 4 (57%) | 7 (100%) |
| Control groups | | | |
| Healthy control | 10 | — | — |
| Disease control | 5 | — | — |

TABLE 5
Comparative reactivity of M. tb H₃₇Ra anti ES-31 antibody and
cocktail antibody (anti ES-31, anti ES-43 and anti EST-6) for
detecting circulating free and immune-complexed antigen in
sputum positive pulmonary tuberculosis by sandwich ELISA.

| | | No. showing positive reaction* for | | | |
|---|---|---|---|---|---|
| | | detection of | | | |
| | | ES-31 Ag | | Cocktail Ag | |
| | No. | Free | | Free | |
| Group | screened | Ag | IC^{$} Ag | Ag | IC^{$} Ag |
| Sputum positive PTB | 68 | 54 (79%) | 62 (91%) | 62 (91%) | 66 (97%) |
| Healthy control | 40 | 2 (5%) | 1 (3%) | 1 (3%) | NR |
| Disease control | 40 | 4 (10%) | 2 (5%) | 3 (8%) | 1 (3%) |
| (COAD^{#} - 12, Bronchial | | | | | |
| asthma - 9, | | | | | |
| Pneumonia - 5, | | | | | |
| PUO^{##} - 10, Pleural | | | | | |
| effusion/empyema - 3, | | | | | |
| Leprosy - 1) | | | | | |

*Sera showing positive reaction at 1:300 dilution
^{$}IC Ag—Immune complexed antigen
^{#}COAD—Chronic obstructive airway diseases
^{##}PUO—Pyrexia of unknown origin
NR—Non-reactive

TABLE 6
Comparative reactivity of M. tb H₃₇Ra anti ES-31 antibody and
cocktail antibody (anti ES-31, anti ES-43 and anti EST-6) for
detecting circulating free and immune-complexed antigen in
different stages of pulmonary tuberculosis by sandwich ELISA

| | | No. showing positive reaction* for detection | | | |
|---|---|---|---|---|---|
| | | of | | | |
| | No. | ES-31 Ag | | Cocktail Ag | |
| Group** | screened | Free Ag | IC^{#} Ag | Free Ag | IC^{#} Ag |
| Fresh cases | 20 | 20 (100%) | 20 (100%) | 20 (100%) | 20 (100%) |
| Chronic cases | 25 | 21 (84%) | 23 (92%) | 20 (80%) | 24 (96%) |
| Relapse cases | 23 | 13 (57%) | 19 (87%) | 22 (96%) | 22 (96%) |

*Sera showing positive reaction at 1:300 dilution
^{#}IC Ag—immune complexed antigen
**The healthy control and non-tubercular disease control were screened for free Ag and IC Ag and the percentage positivity is mentioned in table 4.

TABLE 7
Analysis of free and immune-complexed mycobacterial serine
protease (SEVA TB ES-31) antigen and its antibody in TB with HIV
co-infection.

| | | No. showing Positive reaction for detection of | | | | | |
|---|---|---|---|---|---|---|---|
| | No. of | | | Immune | | Free | Ab/Free |
| | Sera | | Free | complexed | Ab/ | Ag/IC- | Ag/IC- |
| Group | tested | Ab* | Ag** | (IC) Ag** | Free Ag | Ag | Ag |
| TB with | 24 | 11 (46%) | 15 (62%) | 13 (54%) | 19 (79%) | 20 (83%) | 21 (87%) |
| HIV Co- | | | | | | | |
| infection | | | | | | | |

*Sera Dilution 1:600
**Sera Dilution 1:300

TABLE 8
Six months follow up of pulmonary tuberculosis for tuberculous
IgG antibody and circulating tuberculous antigen during
antituberculosis treatment.

| | | | No. showing |
|---|---|---|---|
| | | No. showing | positive reaction |
| | | positive reaction | for Antigen by |
| Day of Sample | | for Antibody | Sandwich |
| collection | No. screened | Indirect ELISA* | ELISA** |
| 0 | 32 | 22 (69%) | 18 (56%) |
| 30 | 32 | 27 (84%) | 17 (53%) |
| 60 | 32 | 28 (87%) | 16 (50%) |
| 180 | 32 | 17 (53%) | 8 (25%) |

*Sera showing positive reaction at 1:200 dilution.
**Sera showing positive reaction at 1:100 dilution.

TABLE 9
Comparative reactivity of M. tuberculosis anti ES-31 antibody and
anti cocktail antigens (anti ES-31, anti ES-43 and anti EST-6)
antibody for detecting circulating free and immunecomplexed (IC)
antigen in HIV-TB patients by sandwich ELISA.

| | | No. showing Positive reaction for detection of | | | | | |
|---|---|---|---|---|---|---|---|
| | | ES-31 Ag | | | Cocktail Ag | | |
| | No. | Free | | | Free | | |
| Group | screened | Ag | IC Ag | Free/IC | Ag | IC Ag | Free/IC |
| TB with HIV | 38 | 21 (55%) | 21 (55%) | 24 (63%) | 23 (61%) | 27 (71%) | 32 (84%) |
| Co-infection | | | | | | | |
| TB | 30 | 20 (67%) | 24 (80%) | 24 (80%) | 25 (83%) | 27 (90%) | 29 (96%) |
| HIV | 26 | 1 (4%) | 0 | 1 (4%) | 2 (8%) | 1 (4%) | 2 (8%) |
| Disease | 25 | 1 (4%) | 0 | 1 (4%) | 3 (12%) | 1 (4%) | 3 (12%) |
| control | | | | | | | |
| Healthy | 28 | 1 (3%) | 0 | 1 (3%) | 1 (3%) | 0 | 1 (3%) |
| control | | | | | | | |

*Sera showing positive reaction at 1:300 dilution

TABLE 10
The usefulness of cocktail of antigens (ES-31 + ES-43 + EST-6) in
antibody & cocktail of antibodies (anti ES-31 + anti ES-43 + anti
EST-6) in antigen detection in sputum/culture positive cases of
extra pulmonary tuberculosis patients.

| | | | | | No. |
|---|---|---|---|---|---|
| | | | | | Showing |
| | | | | | Positive |
| Group sputum | | No. showing positive | | | reaction for |
| +ve/culture | No. of Sera | reaction for detection of | | | Ab/Ag/ |
| +ve cases | tested | Ab* | Ag** | IC-Ag** | IC-Ag |
| EPTB | 32 | 27 (84%) | 15 (47%) | 22 (69%) | 32 (100%) |
| TB Lymphadenopathy | 8 | 6 | 4 | 7 | 8 |
| Tuberculous Meningitis | 5 | 3 | 2 | 4 | 5 |
| Bone & Joint TB | 6 | 6 | 3 | 2 | 6 |
| Abdominal TB | 5 | 4 | 2 | 3 | 5 |
| Pleural TB | 4 | 4 | 2 | 2 | 4 |
| Miliary TB | 4 | 4 | 2 | 4 | 4 |

*Sera Dilution 1:600
**Sera Dilution 1:300

TABLE 11
Detection of tuberculous antibody and circulating antigen (free and IC-
Ag) using SEVA TB ES antigen cocktail III A antigens (ES-31 + ES-43 +
EST-6) or their immunoglobulin cocktail III B in sera of EPTB
suspected by clinical examinations and other tests - a hospital study
during year (January 2004-December 2006)

| | Serum | | | | | |
|---|---|---|---|---|---|---|
| | screened | No. (%) showing positive reaction for | | | | |
| Group | n* | Ab^{†} | Ag^{‡} | CIC-Ag^{‡} | Ag/CIC | Ab/Ag/CIC |
| EPTB^{§} | 164 | 118 (72) | 114 (70) | 104 (63) | 146 (89) | 157 (96) |
| TBLN^{ô} | 35 | 23 (66) | 22 (63) | 25 (71) | 30 (86) | 33 (94) |
| TBM^{¶} | 36 | 25 (69) | 28 (78) | 29 (81) | 33 (92) | 34 (94) |
| Bone and joint TB | 34 | 27 (79) | 24 (71) | 20 (59) | 32 (94) | 33 (97) |
| Abdominal TB | 34 | 24 (71) | 27 (79) | 18 (53) | 30 (88) | 32 (94) |
| Pleural TB | 9 | 5 (56) | 8 (89) | 4 (44) | 9 (100) | 9 (100) |
| Genitourinary TB | 8 | 6 (75) | 3 (38) | 2 (25) | 5 (63) | 8 (100) |
| Miliary TB | 4 | 4 (100) | 1 (25) | 3 (75) | 4 (100) | 4 (100) |
| Ocular TB | 4 | 4 (100) | 1 (25) | 3 (75) | 3 (75) | 4 (100) |
| DISEASE CONTROL | 75 | 9 (12) | 7 (9) | 3 (4) | 7 (9) | 9 (12) |
| Non-specific Lymphadenitis | 13 | 2 (15) | 1 (8) | 0 | 1 (8) | 2 (15) |
| Pyogenic meningitis | 6 | 0 | 0 | 0 | 0 | 0 |
| Seizure | 5 | 0 | 0 | 0 | 0 | 0 |
| Encephalitis | 5 | 1 (20) | 1 (20) | 0 | 1 (20) | 1 (20) |
| Rheumatoid arthritis | 12 | 1 (8) | 1 (8) | 1 (8) | 1 (8) | 1 (8) |
| Ulcerative colitis, | 12 | 0 | 0 | 0 | 0 | 0 |
| Chrohn's disease,, | 4 | 0 | 0 | 0 | 0 | 0 |
| Non-TB pleural effusion | 5 | 1 (20) | 1 (20) | 1 (20) | 1 (20) | 1 (20) |
| Ascitic | 3 | 0 | 0 | 0 | 0 | 0 |
| Nephrotic syndrome, | 2 | 0 | 0 | 0 | 0 | 0 |
| Infertility | 3 | 2 (67) | 1 (33) | 1 (33) | 1 (33) | 2 (67) |
| Iridocyclitis | 1 | 0 | 0 | 0 | 0 | 0 |
| Abdominal abcess | 2 | 1 (50) | 1 (50) | 0 | 1 (50) | 1 (50) |
| Chronic obstructive pulmonary | 2 | 1 (50) | 1 (50) | 0 | 1 (50) | 1 (50) |
| disorder | | | | | | |
| Healthy Control | 75 | 5 (7) | 0 | 0 | 0 | 5 (7) |
| % specificity (compared to | 150 | 91 | 94 | 98 | 94 | 91 |
| Disease and Healthy controls) | | | | | | |

*n = number of patients;
^{†}Sera dilution 1:600;
^{‡}Sera dilution 1:300;
^{§}EPTB = extrapulmonary tuberculosis;
^{ô}TBLN = tuberculous lymphadenopathy;
^{¶}TBM = tuberculous meningitis

TABLE 12
Detection of tuberculous antibody and circulating antigen (free and IC-Ag)
using cocktail III A of antigens (ES-31 + ES-43 + EST-6) and antibody cocktail
III B respectively in confirmed cases of Pulmonary tuberculosis - a hospital
study during January 2004-April 2007

| | Serum | No. showing positive reaction for | | | | |
|---|---|---|---|---|---|---|
| Group | screened n | Ab* | Ag** | IC-Ag** | Ag/IC-Ag | Ab/Ag/IC-Ag |
| PTB | 79 | 73 (92%) | 72 (91%) | 77 (97%) | 77 (97%) | 77 (97%) |
| Disease | 60 | 3 (5%) | 1 (2%) | 1 (2%) | 2 (3%) | 3 (5%) |
| control | | | | | | |
| Healthy | 60 | 2 (3%) | 0 | 1 (2%) | 1 (2%) | 2 (3%) |
| control | | | | | | |
| % | 120 | 96 | 99 | 98 | 97 | 96 |
| Specificity | | | | | | |

PTB = pulmonary tuberculosis;
n = number of patients
*sera dilution 1:600;
**sera dilution 1:300

TABLE 13
Detection of tuberculous antibody and circulating antigen (free and IC-Ag)
using cocktail III A of antigens (ES-31 + ES-43 + EST-6) and antibody
cocktail III B respectively in clinically suspected cases of Pulmonary
tuberculosis - a hospital study during January 2004-April 2007.

| | Serum | No. showing positive reaction for | | | | |
|---|---|---|---|---|---|---|
| Group | screened n | Ab* | Ag** | IC-Ag** | Ag/IC-Ag | Ab/Ag/IC-Ag |
| PTB | 414 | 279 (67%) | 211 (51%) | 255 (62%) | 294 (71%) | 376 (91%) |
| Disease | 60 | 3 (5%) | 1 (2%) | 1 (2%) | 2 (3%) | 3 (5%) |
| control | | | | | | |
| Healthy | 60 | 2 (3%) | 0 | 1 (2%) | 1 (2%) | 2 (3%) |
| control | | | | | | |
| % | 120 | 96 | 99 | 98 | 97 | 96 |
| Specificity | | | | | | |

PTB = pulmonary tuberculosis;
n = number of patients
*sera dilution 1:600;
**sera dilution 1:300

TABLE 14
Detection of tuberculous antibody in contact cases (latent
infection) using SEVA ES-6 antigen.

| | No. | No. showing positive* reaction for | | |
|---|---|---|---|---|
| Group | screened | ES-6 Ag | ES-31 Ag | ES-43 Ag |
| Contact cases | 10 | 4 (40%)** | 1 (10%) | 1 (10%) |
| Healthy control | 20 | 2 (10%) | 2 (10%) | 1 (5%) |

*sera dilution 1:600
**Two contact cases became sputum positive TB after 10 to 15 months

### Summary Table

TABLE 15
Detection of tuberculous antibody/circulating antigen (free and
IC-Ag) in pulmonary, extra-pulmonary and HIV-TB coinfection
using different SEVA TB ES antigen cocktails and their specific
immunoglobulins.

| Antigen/ | | % sensitivity showing positive reaction for | | | | | % Specificity |
|---|---|---|---|---|---|---|---|
| cocktail | Group | Ab* | Ag** | IC-Ag** | Ag/IC-Ag | Ab/Ag/IC-Ag | Ab/Ag/Ic-Ag |
| ES-31 Ag | PTB | 92 | 79 | 91 | 91 | 91 | 92 |
| | Fresh | 76 | 100 | 100 | 100 | 100 | |
| | Relapse | 73 | 57 | 87 | 87 | 87 | |
| | Chronic | 92 | 84 | 92 | 92 | 92 | |
| | EPTB | 77 | 44 | 59 | 59 | 77 | 92 |
| | HIV-TB | 46 | 62 | 54 | 83 | 87 | 95 |
| Cocktail I | PTB | 92 | — | — | — | — | 94 |
| | EPTB | 96 | — | — | — | — | 92 |
| | HIV-TB | — | — | — | — | — | — |
| Cocktail II | PTB | 96 | — | — | — | — | 100 |
| | EPTB | — | — | — | — | — | — |
| | HIV-TB | — | — | — | — | — | — |
| Cocktail III | PTB | 92 | 91 | 97 | 97 | 97 | 92 |
| | Fresh | 75 | 100 | 100 | 100 | 100 | |
| | relapse | 67 | 96 | 96 | 96 | 96 | |
| | Chronic | 93 | 80 | 96 | 96 | 96 | |
| | EPTB | 84 | 47 | 69 | 72 | 100 | 90 |
| | HIV-TB | 55 | 61 | 71 | 84 | 84 | 91 |

Cocktail I:- SEVA ES-31 + ES-41 Ag (better for abdominal & Bone & joint TB)
Cocktail II:- SEVA ES-31 + ES-41 + ES-43 Ag (demonstrated additional bands reacting with additional antigens)
Cocktail III A:- SEVA ES-31 + ES-43 + EST-6 (ES-38 & ES-41) Ag or their specific immunoglobulins (cocktail III B)
Ab = antibody,
Ag = Antigen,
IC-Ag = immune complexed (bound) antigen.
PTB = pulmonary tuberculosis;
EPTB = extrapulmonary tuberculosis;
n = number of patients;
= sera dilution 1:600;
**= sera dilution 1:300;
— = study not done
Note:
Number and patients differ in screening of cocktail antigens/antibodies.
Innovations:
1. Development of antigen cocktail III A for antibody detection by Indirect ELISA (Assay 1)
2. Development of antibody cocktail III B for antigen and immune complexed antigen detection by Sandwich ELISA (Assay 2)
3. Antigen and immune complexed antigen in HIV-TB co-infection using antibody cocktail III B by ELISA.

TABLE 16
Biochemical Characterization of ES-31, ES-41 & ES-43
Antigens

| | Antigen activity reactive titer* | | |
|---|---|---|---|
| Treatment | ES-31 | ES-41 | ES-43 |
| Untreated | 1000 | 1000 | 1000 |
| Heat (100° C., 30 min) | 10 | 10 | 10 |
| Enzymes | | | |
| Trypsin | 0 | 0 | 0 |
| Chymotrypsin | 1 | — | — |
| Pronase | 0 | 0 | 0 |
| Pepsin | 0 | 0 | 0 |
| Lipase | 1000 | 0 | 1000 |
| Amylase | 0 | — | — |
| Glycanase N—F | 0 | — | 0 |
| Sodium meta periodate | 0 | 1000 | 10 |

*The reciprocal of the highest dilution of fraction showing positive reaction. The initial protein concentration of each fraction was 200 μg/ml.

TABLE 17
Purification of Serine protease (SEVA TB ES-31
antigen) from M. tuberculosis H37Ra culture fluid.

| | Total | Total | Specific | | |
|---|---|---|---|---|---|
| | Protein | Activity | Activity* | Purification | Yield |
| Fraction | (mg) | (U) | (U/mg) | Factor | (%) |
| M. tb. H₃₇Ra culture | 333 | 0.672 × 10⁻³ | 2.0187 × 10⁻⁶ | 1 | 100 |
| filtrate | | | | | |
| Excretory-secretory | 20 | 0.328 × 10⁻³ | 16.4 × 10⁻⁶ | 8.12 | 49 |
| (ES) Ag | | | | | |
| Ammonium sulphate | 4 | 0.258 × 10⁻³ | 64.58 × 10⁻⁶ | 32 | 38 |
| Precipition (ESAS-Ag) | | | | | |
| SDS-PAGE fractionation | 0.21 | 0.225 × 10⁻³ | 1071 × 10⁻⁶ | 531 | 33.5 |
| (ESAS-7 Ag) | | | | | |
| FPLC fraction | 0.028 | 0.175 × 10⁻³ | 6250 × 10⁻⁶ | 3096 | 26 |
| ‘S’ cation exchange | | | | | |
| column | | | | | |
| (ESAS-7F Ag) | | | | | |

*Unit of activity is A₄₄₀ × 1000/mg protein/min.

TABLE18
Effect of metallo serine protease inhibitors on
mycobacterial serine protease (Seva TB ES-31)

| | | | Residual | Inhibition |
|---|---|---|---|---|
| Inhibitors | Concentration | | activity (%) | (%) |
| Control | — | | 100 | 0 |
| Pefabloc | 0.5 | mM | 7.56 ± 0.07 | 92 |
| 3,4-Dichloroisocoumarin | 10 | μM | 9.84 ± 0.07 | 90 |
| Phenyl methyl sulphonyl | 1 | mM | 19.6 ± 0.2 | 80 |
| fluoride (PMSF) | | | | |
| Ethylene diamine tetracetic | 1 | mM | 35.3 ± 0.89 | 65 |
| acid (EDTA) | | | | |
| 1,10 Phenanthroline | 0.5 | mM | 22.4 ± 0.47 | 78 |
| 1,10 Phenanthroline + | 1 | mM | 92.21 ± 0.03 | 8 |
| ZnSO₄ | | | | |

Conclusion: SEVA ES-31 antigen is a zinc containing serine protease

TABLE 19
Effect of antitubercular drugs on mycobacterial serine protease
(SEVA TB ES-31 Ag) and M. tb H₃₇Ra bacilli in culture
(LJ slant/Sauton's medium)

| | | | | Effect on H₃₇Ra |
|---|---|---|---|---|
| | | % Inhibition | MIC (mg/l)/ | culture* |
| | Concentration | of Serine | Resistance | (LJ slant/Sautons' |
| Drugs | (mg/ml) | protease | Ratio | medium) |
| Control | — | 0 | 0 | Growth |
| RMP | 150 | 0 | >64 | — |
| INH | 75 | 85 | >1 | No Growth |
| EMB | 275 | 70 | >8 | No Growth |
| PZA | 400 | 0 | >100 | — |
| SM | 400 | 30 | Resistance | No Growth |
| | | | ratio > 8 | |

MIC is minimum inhibitory concentration (mg/l) that inhibits microbial growth
Optical density of culture on Sautons'broth was measured at 540 nm.
RMP = rifampicin;
INH = isoniazid;
EMB = ethambutol;
SM = streptomycin

TABLE 20
Effect of serine protease inhibitors on H₃₇Ra bacilli in
culture (Sauton's medium/LJ slant)

| Inhibitor | Concentration (μM) | | % Inhibition on culture |
|---|---|---|---|
| Control | 0 | | 0 |
| Pefabloc | 10 | μM | 88 |
| 3,4-Dichloroisocoumarin | 0.2 | μM | 85 |
| Phenyl Methyl Sulphonyl | 20 | μM | 72 |
| Fluoride (PMSF) | | | |
| 1,10 Phenanthroline | 10 | μM | 81 |

*Inhibitors showed inhibition of bacillary growth on Lowenstein-Jensen slant at the same concentration.

1. A cocktail of two or more of substantially pure mycobacterial excretory secretory protein/polypeptide antigens, affinity purified glycoprotein or lipoprotein in nature with approximate molecular size of 31, 41, 43 and 38 kDa (examples 3, 4 & 5) (Seva ES-31, Seva ES-41, Seva ES-43 and Seva EST-6) or combination of immunogenic portion of these polypeptide sequences by recombinant or chemically synthesized, for use as a pharmaceutical or diagnostic reagent.

2. A diagnostic reagent for antibody assay, comprising one or more substantially pure mycobacterial polypeptides or immunogenic portion of any one of the peptide sequences from Seva ES-31, Seva ES-41, Seva ES-43 and Seva EST-6 for use in diagnosis of different stages (Fresh, chronic and relapse) of tuberculosis in adults and children.

3. DNA fragments in isolated form, which comprise one or more nucleic acid sequences, which encode immunogenic portion of polypeptide or recombinant antigens as defined in claim 1. with potential for diagnosis of tuberculosis.

4. A diagnostic tool with high specificity for host immune response independent antigen assay, comprising one or more monoclonal or polyclonal (affinity purified) antibodies which are reactive with a polypeptide in claim 1 for use in diagnosis of pulmonary mycobacterium tuberculosis.

5. A diagnostic tool for antigen assay, comprising one or more monoclonal or polyclonal (affinity purified) antibodies which are reactive with a polypeptide in claim 1 for use in diagnosis of MDR tuberculosis, XDR tuberculosis and tuberculosis with HIV co-infection.

6. A diagnostic tool for antigen assay, comprising one or more monoclonal or polyclonal antibodies as in claim 5 for immunomonitoring during antitubercular treatment for assessment of compliance or drug resistance.

7. A diagnostic tool for antibody assay comprising one or more substantially pure mycobacterial polypeptides in claim 1 for use in antibody detection and one or more monoclonal or polyclonal (affinity purified) antibodies which are reactive with a polypeptide in claim 1 for use in antigen detection in diagnosis of extrapulmonary tuberculosis such as TB meningitis, lymphnode, bone and joint, abdominal, miliary, genitourinary, pleural and ocular tuberculosis.

8. A combination of antigens in ELISA (indirect, capture or immune complex) or rapid immunological test according to claim 1 where in said antigens are derived from mycobacterium bacilli culture filtrate or bacilli or produced by recombinant technology.

9. A combination of monoclonal or affinity purified monospecific antibodies in ELISA (indirect, capture or immune complex) or rapid format immunological test or by ultrasensitive immuno PCR as per claim 4 obtained from serum from animal or human.

10. ELISA based Indirect and Sandwich assay systems for detection of antibody, antigen and immune complexed antigen for the diagnosis and monitoring of pulmonary and extrapulmonary tuberculosis in adults and children and TB with HIV coinfection.

11. A drug target for screening anti tubercular drugs comprising Seva TB ES-31.

12. A drug target according to claim 11, wherein the drugs comprise of Antitubercular drugs namely Isoniazid, Ethambutol and Streptomycin.


SEQUENCE LISTING (2 sequences)

SEQ ID NO: 1 - DNA, 747 bp, Mycobacterium tuberculosis
  CDS 1..747<s300>
aacaagcaaaaagcgttagctgcagcactgggccagattgaaaagcaattcggtaaaggctccatcatgcgtctgggtgaagatcgctctatggacgtcgaaacgatctctaccggctctttgtctttggatatcgcgttaggtgctggcggcttgccaatgggccgtatcgttgaaatttatggtccggaatcctccggtaaaaccacgctgactctgcaggttatcgctgctgcacagcgtgaaggcaaaacctgtgcgttcatcgatgcagaacacgcgctggaccctatctacgctaaaaaactgggcgtcgatatcgataacctgctgtgttctcagccagataccggtgaacaggctctggaaatctgtgatgcgctgacccgttcaggcgccgttgacgttatcatcgttgactccgttgccgcactgacgccaaaagctgaaatcgaaggcgaaattggtgactctcacatgggcctcgcagctcgtatgatgagccaggcgatgcgtaagcttgcgggtaacctgaaaaacgccaataccctgctgatcttcatcaaccagatccgtatgaaaatcggtgtgatgttcggtaacccggaaaccaccaccggcggtaacgccctgaaattctatgcctctgtacgtctggatatccgtcgtatcggcgctatcaaagaaggtgatgtggttgtcggtagtgaaacacgtgtgaaagtcgtgaagaacaaaatcgccgca

SEQ ID NO: 2 - protein, 249 aa, Mycobacterium tuberculosis
NKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYAKKLGVDIDNLLCSQPDTGEQALEICDALTRSGAVDVIIVDSVAALTPKAEIEGEIGDSHMGLAARMMSQAMRKLAGNLKNANTLLIFINQIRMKIGVMFGNPETTTGGNALKFYASVRLDIRRIGAIKEGDVVVGSETRVKVVKNKIAA